(19) United States
(12) Patent Application Publication
(10) Pub. No.: US 2025/0001049 A1
(43) Pub. Date: Jan. 2, 2025

(54) BULKING AND COLLAGEN STIMULATING HYDROGEL COMPOSITIONS

(71) Applicant: Prohibix LLC, Philadelphia, PA (US)

(72) Inventor: Brendan Purcell, Boulder, CO (US)

(73) Assignee: Prohibix LLC, Philadelphia, PA (US)

(21) Appl. No.: 18/701,151

(22) PCT Filed: Oct. 17, 2022
(86) PCT No.: PCT/US2022/046854

§ 371 Date: Apr. 12, 2024

Related U.S. Application Data
(60) Provisional application No. 63/256,112, filed on Oct. 15, 2021.

(51) Int. Cl. A61L 27/52; A61L 27/20

(52) U.S. Cl. CPC A61L 27/52; A61L 27/20; A61L 2300/412; A61L 2430/34

(57) ABSTRACT

Hydrogel compositions comprised of multiple fractions of collagen stimulatory agents are disclosed. The compositions may enable continuous bulking through a unique combination of hydrogel temporal mechanics and collagen production. A composition may include at least one fraction of hydrogels combined with at least one collagen stimulatory agent. The at least one fraction may be a plurality of fractions.

### CROSS REFERENCE TO RELATED APPLICATION

This application claims the benefit of U.S. provisional application No. 63/256,112, which was filed Oct. 15, 2021, was titled BULKING AND COLLAGEN STIMULATING HYDROGEL COMPOSITIONS, and is incorporated herein by reference, as if fully set forth.

### FIELD

This relates to hydrogels compositions that bulk tissue through a combination of controlled mechanics and presentation of collagen stimulating agents.

### BACKGROUND

Hydrogels are water swollen networks of polymers held together by physical or chemical bonds. The polymer concentration along with extent of polymer crosslinking determine the crosslink density of a hydrogel. Hydrogel crosslink density can be tuned to achieve a range of mechanical properties, swelling and stabilities.

Hyaluronic acid (HA) is a linear glycosaminoglycan comprised of repeating D-glucuronic acid and N-acetyl-D-glucosamine disaccharides. It is a hydrophilic, water-swollen polymer, and is abundant in the extracellular matrix of tissues throughout the body. HA swelling has important mechanical and biological roles in regulating tissue hydration and mechanics, tissue formation, wound healing, and inflammation. HA polymers can be found in the body at molecular weights ranging from 5,000 daltons (Da) to 20,000,000 Da. HA dissolved in aqueous buffers form shear-thinning solutions with viscoelastic properties that are directly proportional to HA concentration and molecular weight. See Snetkov, P., et al., Hyaluronic Acid: The Influence of Molecular Weight on Structural, Physical, Physico-Chemical, and Degradable Properties of Biopolymer. Polymers (Basel), 2020. 12 (8), which is incorporated herein by reference, as if fully set forth.

Crosslinks can be introduced between dissolved HA polymers to form hydrogels for increased stability in the body. See Burdick, J. A. and G. D. Prestwich, Hyaluronic acid hydrogels for biomedical applications. Adv Mater, 2011. 23 (12): p. H41-56, which is incorporated herein by reference, as if fully set forth. Crosslinked HA hydrogels are commonly administered via intradermal or subcutaneous injection to provide volume under the skin for aesthetic manipulations. These products are typically comprised of HA crosslinked with 1,4-Butanediol diglycidyl ether (BDDE) through a condensation reaction under elevated temperature and basic pH. By controlling the extent of BDDE crosslinking, viscous HA solutions and hydrogels can be fabricated with a range of physical properties. For example, a range of BDDE crosslinked HA dermal filler products have been developed with a different swelling, stiffness and tissue distribution characteristics. See La Gatta, A., et al., Hyaluronan-based hydrogels as dermal fillers: The biophysical properties that translate into a “volumetric” effect. Plos One, June 2019, which is incorporated herein by reference, as if fully set forth. In addition, these products have shown a range of persistence in the skin. See Hillel, A. T., et al., Validation of a Small Animal Model for Soft Tissue Filler Characterization. Dermatologic Surgery, March 2012, 38:3, which is incorporated herein by reference, as if fully set forth.

While some studies suggest that HA hydrogel based dermal fillers stimulate skin fibroblasts to produce collagen (See La Gatte et al. and Wang, F., et al., In Vivo Stimulation of De Novo Collagen Production Caused by Cross-linked Hyaluronic Acid Dermal Filler Injections in Photodamaged Human Skin, Arch Dermatol. 2007; 143:155-163, which is incorporated herein by reference, as if fully set forth), it does not seem to be enough to maintain the hydrogel volume after the hydrogel has been degraded. Alternatively, injection of microparticle products based on poly(L-lactic acid), poly(caprolactone) or hydroxyapatite have been shown to stimulate a macrophage response that results in the gradual production of a collagen network in the skin. See Stein, P., et al., The biological basis for poly-L-lactic acid-induced augmentation, Journal of Dermatological Science 78 (2015) 26-33, which is incorporated herein by reference, as if fully set forth. Some products, such as those based on poly(L-lactic acid) (PLLA), require multiple injections spaced weeks apart to achieve adequate collagen production and achievement of aesthetic manipulation. For example, Sculptra (Galderma) is indicated for up to 4 injections, spaced 3 weeks apart. A viscous carboxymethylcellulose carrier is used to achieve a uniform suspension of PLLA particles after reconstitution and prior to injection, but quickly dissipates following injection.

Hydrolytically degradable HA hydrogels have been explored as a carrier for poly(lactic-co-glycolic acid) (PLGA) microparticles to provide an initial bulking period [See Tous, E., et al., Tunable Hydrogel-Microsphere Composites that Modulate Local Inflammation and Collagen Bulking, Acta Biomater. 2012 September; 8 (9): 3218-3227, which is incorporated herein by reference, as if fully set forth], however hydrogel stabilities of less than 30 days were required for PLLA presentation and collagen production.

### SUMMARY

In an aspect, the invention relates to a composition comprising a first fraction of hydrogel microparticles. The hydrogel microparticles comprise an encapsulated collagen stimulatory agent.

In an aspect, the invention relates to a method of producing and maintaining an aesthetic feature. The method comprises injecting a composition comprising a first fraction of hydrogel microparticles into the skin of a subject. The hydrogel microparticles comprise an encapsulated collagen stimulatory agent.

In an aspect, the invention relates to a method of producing or maintaining a volume in or under the skin. The method comprises administering a composition comprising a first fraction of hydrogel microparticles to the skin of a subject.

In an aspect, the invention relates to a use of a composition comprising a first fraction of hydrogel microparticles for producing and maintaining an aesthetic feature of the skin of a subject.

In an aspect, the invention relates to a use of a composition comprising a first fraction of hydrogel microparticles for producing or maintaining a volume in or under the skin of a subject.

In an aspect, the invention relates to a composition comprising hydrogel microparticles with encapsulated collagen stimulatory agent and greater than 30 days stability.

In an aspect, the invention relates to a composition comprising hydrogel microparticles with encapsulated collagen stimulatory agent and a collagen stimulatory agent mixed with the hydrogel microparticles.

In an aspect, the invention relates to a composition comprising a first fraction of hydrogel microparticle with encapsulated collagen stimulatory agent and a second fraction of hydrogel microparticle with encapsulated collagen stimulatory agent, wherein hydrogel fractions have different stabilities.

In an aspect, the invention relates to a composition comprising a first fraction of collagen stimulatory agent and a second fraction of collagen stimulatory agent, wherein at least one fraction is encapsulated in a hydrogel.

In an aspect, the inventions related to a composition comprising a first fraction of collagen stimulatory microparticle and a second fraction of collagen stimulatory microparticle, wherein at least one fraction is encapsulated in a hydrogel.

In an aspect, the invention relates to a method of providing producing and maintaining an aesthetic feature. The method comprises injecting any one or more composition herein into skin of a subject.

In an aspect, the invention relates to a composition comprising any composition or combination of compositions described herein.

In an aspect, the invention relates to a method of making comprising any method of making described herein.

In an aspect, the invention relates to a method of treating comprising administering any one or more composition herein to a subject.

### BRIEF DESCRIPTION OF THE DRAWINGS

The following detailed description of the preferred embodiment of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1 shows a continuous bulking volume in the skin through a combination of early hydrogel mechanics followed by collagen production. Multiple fractions of HA hydrogels that degrade at different rates are required to maintain the bulking volume during the process of collagen production as collagen production is stimulated by release of an agent which is only achieved through loss of hydrogel mechanical stability. Therefore, when one fraction of hydrogel loses stability and releases collagen stimulatory agents to begin collagen production, there is not a noticeable loss of the aesthetic feature.

FIG. 2 shows the swelling ratio (hydrogel mass-initial hydrogel mass)/(initial hydrogel mass) of HA hydrogels produced with acrylate modified HA and dithiothreitol (DTT) crosslinker at Low (4% acrylate modification), Medium (9% acrylate modification) and High (20% acrylate modification) crosslink densities (n=3-4 per group). Hydrogels were incubated in a 10× volume of PBS at 37° C. The hydrogels could no longer be recovered from the buffer after the last data point shown and therefore considered completely degraded.

FIG. 3 shows the volume (A) and hydroxyproline content (B) of explanted tissue masses 84 days following subcutaneous injection of 0.1 mL of 2 wt % HA hydrogel microparticles with 7.5 wt % PLLA microparticles encapsulated. The HA Gel Low group was comprised of microparticles produced from 4% acrylate modified HA and DTT crosslinker while the HA Gel Combo group was comprised of three fractions of microparticles produced from 4%, 9% and 20% acrylate modified HA and DTT crosslinker mixed at equal mass ratios. P values from a student's t-test are shown to compare volumes and hydroxyproline content between groups with p<0.05 considered statistically significant (n=5 for HA Gel Low and n=7 for HA Gel Combo).

### DETAILED DESCRIPTION

Certain terminology is used in the following description for convenience only and is not limiting. The words “right,” “left,” “top,” and “bottom” designate directions in the drawings to which reference is made.
“Hydrogel” is a water swollen network of polymers.
“Crosslink” refers to covalent or non-covalent bonds formed between polymers.
“Microparticle” refers to a particle with an average outer dimension between 1 and 2,000 microns. Microparticles may have dimensions greater than 1,000 microns. Average outer dimension can be calculated by taking the average of at least 2 measurements drawn between opposite sides of a particle, where the measurements are taken along or parallel to the longer axis of the particle and along or parallel to the shorter axis of the particle.
“Crosslinker” refers to molecules that form bonds between polymers. Crosslinkers can react with polymer to form a crosslinking bond or be chemically modified onto the polymer prior to crosslinking to other polymers.
“Hydrolytically degradable” refers to a crosslink that contains covalent bonds that dissociate due to nucleophilic attack of a water molecule.
“Extent of chemical modification” or “percent modification” refers to the percent of repeat units of a polymer that are modified with a new chemical group. For HA, the repeat unit is the disaccharide composed of D-glucuronic acid and N-acetyl-D-glucosamine, linked via alternating β-(1→4) and β-(1→3) glycosidic bonds.
“Extent of crosslinking” refers to the percent of repeat units on a polymer that are bound to another polymer or linker molecule through covalent or non-covalent interactions. For HA, the repeat unit is the disaccharide composed of D-glucuronic acid and N-acetyl-D-glucosamine, linked via alternating β-(1→4) and β-(1→3) glycosidic bonds.
A “Fraction” is a subset of materials produced by certain chemical synthesis or formulation process steps. Fractions are subsets of materials where each subset is produced through chemical synthesis or formulation process steps separately than the other subsets. For example, PLLA encapsulated in a hydrogel microparticle and PLLA mixed with the PLLA encapsulated hydrogel microparticles are different fractions of PLLA. In the context of hydrogel microparticles, a fraction is a subset of particles that can be identified by differences in molecular weight, extent or type of chemical crosslinking, extent or type of chemical modification, crosslink density, size or stability. These subsets may have a distribution around these properties but can be identified through a non-zero difference in the means of their distributions. For example, hydrogel microparticles fabricated with 10% extent of chemical crosslinking and hydrogel microparticles fabricated with 30% extent of chemical crosslinking are different fractions of hydrogel.
“Drug” is any substance used in the diagnosis, treatment, or prevention of a disease or pathology, or as a component of a medication.
“Molecular weight” is a measure of the size of a polymer. As used herein, the term “molecular weight” refers to the number average molecular weight of a polymer as determined by gel permeation chromatography in aqueous buffer. When used to describe an HA polymer, molecular weight refers to the linear uncrosslinked HA.
“Stability” is a measure of how long a hydrogel remains insoluble, i.e., able to be separated from its surrounding fluid with a spatula or through centrifugation at 2,000×g for 10 min. Stability can be measured by incubating a hydrogel in 10× or more volume of aqueous buffer such as phosphate buffered saline at pH 7.4 and 37° C. and measuring swelling ratios until the hydrogel can no longer be separated from the buffer. At this point the hydrogel is considered completely dissolved or degraded.
“Storage modulus” or (G′) is a measure of stored energy under force and represents the elastic portion of the complex modulus of viscoelastic materials. G′ can be determined by oscillatory rheology.
“Loss modulus” or (G″) is a measure of energy dissipation under force and represents the viscous portion of the complex modulus of viscoelastic materials. G″ can be determined by oscillatory rheology.
“Physiological buffer” is an aqueous buffer with a pH and ionic concentrations found in the body. Phosphate buffered saline (PBS) which comprises 137 mM NaCl, 2.7 mM KCl, 10 mM Na2HPO4, 1.8 mM KH2PO4, pH 7.4 is a commonly used physiological buffer.
“Physiological conditions” refers to pH 7.4 and 37° C.
The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

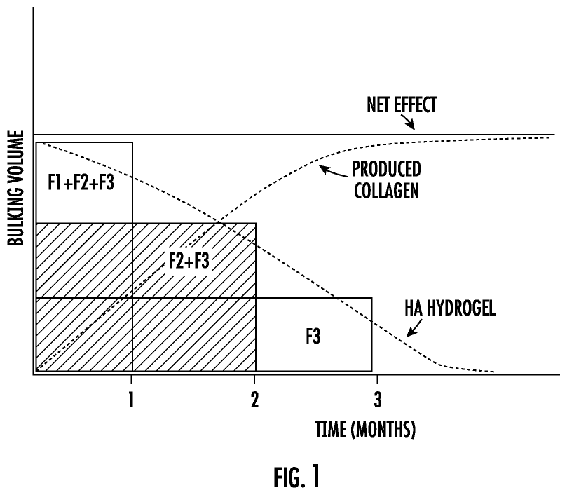

An embodiment comprises a composition comprising at least one fraction of hydrogels combined with at least one collagen stimulatory agent. The at least one fraction may be a plurality of fractions. The at least one fraction may be multiple fractions. The at least one fraction may be 1, 2, 3, 4, 5, 6, 7, 8, 9, 10, 11, 12, 13, 14, 15, 16, 17, 18, 19, or 20 fractions, or more 1, 2, 3, 4, 5, 6, 7, 8, 9, 10, 11, 12, 13, 14, 15, 16, 17, 18, 19, or 20 that fractions, a number of fractions between any two of 1, 2, 3, 4, 5, 6, 7, 8, 9, 10, 11, 12, 13, 14, 15, 16, 17, 18, 19, or 20 fractions. Embodiments include compositions wherein the multiple fractions are uniformly mixed. The composition may gradually stimulate collagen production. The composition may maintain the initial bulking volume after injection into the body. The maintenance of initial bulking volume may occur while collagen production is stimulated. Each fraction may have a different hydrogel stability. Each fraction may release collagen stimulatory agent at a different rate. See FIG. 1.

An embodiment comprises a method of creating or maintaining an aesthetic feature. The method may maintain the aesthetic feature through a combination of hydrogel mechanics and endogenous collagen production. The method may comprise injecting a composition herein to a site where the aesthetic feature is to be created or maintained.

The collagen stimulatory agents may be one or more of encapsulated in the hydrogels microparticles or mixed on the outside of the hydrogel microparticles.

There may be one fraction of hydrogel microparticles. There may be more than one fraction of hydrogel microparticles with different stabilities in the body. There may be 1, 2, 3, 4, 5, or more fractions of hydrogel microparticles.

A composition herein may be used to provide an aesthetic feature in a subject. The composition may be injected near, in, or under a site where the aesthetic feature is to be created. There may be enough fractions of hydrogels such that when one fraction is no longer stable or present, its absence does not alter the aesthetic feature.

An embodiment comprises a method of providing an aesthetic feature in a subject. The method may comprises injecting a composition herein near, in, or under a site where the aesthetic feature is to be created. There may be enough fractions of hydrogels such that when one fraction is no longer stable or present, its absence does not alter the aesthetic feature.

An embodiment comprises a use of a composition herein for providing an aesthetic feature in a subject. The use may comprises injecting a composition herein near, in, or under a site where the aesthetic feature is to be created. There may be enough fractions of hydrogels such that when one fraction is no longer stable or present, its absence does not alter the aesthetic feature.

Aesthetic features may comprise added volume in or under the skin. This feature may reduce the appearance of wrinkles or increase the projection of tissues including but not limited to the lips, cheeks, jaw line, brow, nasolabial folds, back, shoulder blades, hands, and buttocks. The aesthetic feature may be an enhancement of naturally occurring tissues or may be a replacement for the loss of tissues due to disease, injury or surgery.

The collagen stimulatory agents may be or be part of microparticles. The microparticles may be but are not limited to poly(L-lactic acid), poly(lactic-co-glycolic acid), polycaprolactone, or hydroxyapatite. The microparticles may be spheres. The microparticles may be rods or fibers.

The collagen stimulatory agents may be or be part of nanoparticles. The nanoparticles may be but are not limited to gold. The nanoparticles may be spheres. The nanoparticles may be rods or fibers.

The collagen stimulatory agents may comprise one or more drugs.

The one or more drugs may be selected from antioxidants, collagen stimulatory peptides, growth factors or peptide sequences derived from growth factors, cytokines, and molecules derived from human blood. The one or more drug may be selected from small molecules, peptides, cytokines, proteins, polysaccharides, synthetic polymers, particles, DNA plasmids, mRNA, cells, cellular exosomes and other cellular components, plasma or plasma derived molecules. The one or more drug may be a reducing agent.

The antioxidants may be at least one of glutathione, ascorbic acid, ascorbic acid palmitate, ascorbic acid glucoside, or other modified ascorbic acid, tocopherol, fatty acids, vitamins, vitamin A, or retinol.

The collagen stimulatory peptides may contain amino acid sequences similar to collagen. The peptides may be collagen-like peptides.

The growth factors or peptide sequences derived from growth factors may stimulate collagen production. The growth factors include but are not limited to fibroblast growth factors 1-23, insulin-like growth factors, transforming growth factor beta 1-3, platelet derived growth factors, epidermal growth factors, hepatocyte growth factors, connective tissue growth factor.

The cytokines include but are not limited one or more interleukin.

The molecules derived from human blood may be derived from platelet rich plasma.

The one or more drug may be encapsulated during hydrogel crosslinking. The collagen stimulatory agents may be encapsulated by reconstituting the hydrogel microparticle powder with a solution of the drug prior to administration.

A composition herein may comprise one or more drugs. The one or more drugs may stimulate collagen production.

The one more drug may be encapsulated at a total concentration greater than 1 picomolar, 1 nanomolar, 1 micromolar or 1 millimolar. The drug may be encapsulated at concentrations between 1 picomolar, 1 nanomolar, 1 micromolar, 1 millimolar or 100 millimolar, or any one of 1 picomolar, 1 nanomolar, 1 micromolar, 1 millimolar or 100 millimolar.

The composition may be a powder. The powder may be reconstituted prior to injection.

The hydrogel may comprise polymers known in the art including but not limited to polysaccharides, carboxymethylcellulose, alginate, polyethylene glycol, and hyaluronic acid.

The polymers may have polymer molecular weights of greater than 10 KDa, 100 kDa, 300 KDa, 500 KDa, 700 KDa, 1000 KDa, 2000 KDa, 3000 KDa, or 4000 KDa, in a range between and including any two of 10 KDa, 100 kDa, 300 KDa, 500 KDa, 700 KDa, 1000 KDa, 2000 KDa, 3000 KDa, or 4000 KDa, or one of 10 KDa, 100 kDa, 300 KDa, 500 KDa, 700 KDa, 1000 KDa, 2000 KDa, 3000 KDa, or 4000 KDa.

The polymer may be at concentrations greater than 0.5 wt %, 1 wt %, 2 wt %, 3 wt %, 4 wt %, or 5 wt %, in a range between and including any two of 0.5 wt %, 1 wt %, 2 wt %, 3 wt %, 4 wt %, 5 wt %, or 10 wt %, or one of 0.5 wt %, 1 wt %, 2 wt %, 3 wt %, 4 wt %, or 5 wt %, or 10 wt %.

The polymer may be crosslinked. The extent of chemical crosslinking may be greater than 0.1%, 0.5%, 1%, 2%, 3%, 4%, 5%, 6%, 7%, 8%, 9%, 10%, 11%, 12%, 13%, 14%, 15%, 20%, 30%, 40%, 50%, 60%, 70%, 80%, or 90% in a range between and including any two of 0.1%, 0.5%, 1%, 2%, 3%, 4%, 5%, 6%, 7%, 8%, 9%, 10%, 11%, 12%, 13%, 14%, 15%, 20%, 30%, 40%, 50%, 60%, 70%, 80%, 90% or 100%, or one of 0.1%, 0.5%, 1%, 2%, 3%, 4%, 5%, 6%, 7%, 8%, 9%, 10%, 11%, 12%, 13%, 14%, 15%, 20%, 30%, 40%, 50%, 60%, 70%, 80%, 90% or 100%.

The hydrogels of a composition herein may have two or more fractions with storage moduli greater than 10 Pa, 100 Pa, 200 Pa, 300 Pa, 400 Pa, 500 Pa, 600 Pa, 700 Pa, 800 Pa, 900 Pa, 1 kPa, or 10 kPa in a range between and including any two of 10 Pa, 100 Pa, 200 Pa, 300 Pa, 400 Pa, 500 Pa, 600 Pa, 700 Pa, 800 Pa, 900 Pa, 1 kPa, 2 kPa, or 10 kPa, or any one of 10 Pa, 100 Pa, 200 Pa, 300 Pa, 400 Pa, 500 Pa, 600 Pa, 700 Pa, 800 Pa, 900 Pa, 1 kPa, 10 kPa, or 100 kPa.

Compositions herein may comprise hydrogel fractions that are particles. Embodiments include hydrogel particles with an average outer dimension greater than 50 μm, 100 μm, 200 μm, 300 μm, 400 μm, 500 μm, 600 μm, 700 μm, 800 μm, 900 μm, 1 mm or 2 mm, in a range between and including any two of 50 μm, 100 μm, 200 μm, 300 μm, 400 μm, 500 μm, 600 μm, 700 μm, 800 μm, 900 μm, 1 mm or 2 mm, or any one of 50 μm, 100 μm, 200 μm, 300 μm, 400 μm, 500 μm, 600 μm, 700 μm, 800 μm, 900 μm, 1 mm or 2 mm.

Crosslinks of one or more hydrogel microparticle fraction may comprise 1,4-butanediol diglycidyl ether. Crosslinks of one or more hydrogel microparticle fraction may have hydrolytically degradable chemistries. The hydrolytically degradable chemistries may be selected from one or more of lactic acid, poly-L-lactic acid, caprolactone, polycaprolactone, glycolic acid, polyglycolic acid, hydroxyethyl methacrylate, anhydrides, or ester linkages.

The polymers may be chemically modified polymers. The chemical modification may comprise at least one of an aldehyde, hydrazide, thiol, acrylate, methacrylate, hydroxyethylmethacrylate, norbornene, azide, alkyne, glycidyl methacrylate, haloacetate, benzyl ester, tyramide, glycidyl ether, epoxide, cyclodextrin, adamantane, moieties conjugated through an ester bond, or hydrolytically degradable moieties. The hydrolytically degradable moieties may be selected from one or more of lactic acid, poly-L-lactic acid, caprolactone, polycaprolactone, glycolic acid, polyglycolic acid, hydroxyethyl methacrylate, and anhydrides.

The extent of chemical modification may be greater than 0.1%, 0.5%, 1%, 2%, 3%, 4%, 5%, 6%, 7%, 8%, 9%, 10%, 11%, 12%, 13%, 14%, 15%, 20%, 30%, 40%, 50%, 60%, 70%, 80%, or 90% in a range between and including any two of 0.1%, 0.5%, 1%, 2%, 3%, 4%, 5%, 6%, 7%, 8%, 9%, 10%, 11%, 12%, 13%, 14%, 15%, 20%, 30%, 40%, 50%, 60%, 70%, 80%, 90% or 100%, or one of 0.1%, 0.5%, 1%, 2%, 3%, 4%, 5%, 6%, 7%, 8%, 9%, 10%, 11%, 12%, 13%, 14%, 15%, 20%, 30%, 40%, 50%, 60%, 70%, 80%, 90% or 100%.

The hydrogels may persist in the skin for greater than 1 day, 2 days, 3 days, 7 days, 14 days, 21 days, 28 days, 1 month, 2 months, 3 months, 4 months, 5 months, 6 months, 7 months, 8 months, 9 months, 10 months, 11, months or 12 months, in a range between and including any two of 1 day, 2 days, 3 days, 7 days, 14 days, 21 days, 28 days, 1 month, 2 months, 3 months, 4 months, 5 months, 6 months, 7 months, 8 months, 9 months, 10 months, 11, months or 12 months, or one of 1 day, 2 days, 3 days, 7 days, 14 days, 21 days, 28 days, 1 month, 2 months, 3 months, 4 months, 5 months, 6 months, 7 months, 8 months, 9 months, 10 months, 11, months or 12 months.

A composition herein may have one hydrogel fraction, more than one hydrogel fraction. A composition herein may have on more than two, three, four, five, six, seven, or eight fractions, or a number of fractions in a range between and including any two of two, three, four, five, six, seven, or eight fractions, or any one of two, three, four, five, six, seven, or eight fractions.

A hydrogel fraction may encapsulate microparticles. One or more hydrogel fractions in a composition may encapsulate microparticles. Microparticles may be mixed with hydrogel fractions. The microparticles may comprise one or more agents known to stimulate collagen production. The agents may be but are not limited to poly(L-lactic acid), poly(lactic-co-glycolic acid), polycaprolactone, or hydroxyapatite. The microparticle(s) may have an average outer dimension greater than 1 μm, 10 μm, 20 μm, 30 μm, 40 μm, 50 μm, 60 μm, 70 μm, 80 μm, 90 μm, 100 μm, or 150 μm, in a range between and including any two of 1 μm, 10 μm, 20 μm, 30 μm, 40 μm, 50 μm, 60 μm, 70 μm, 80 μm, 90 μm, 100 μm, or 150 μm, or any one of 1 μm, 10 μm, 20 μm, 30 μm, 40 μm, 50 μm, 60 μm, 70 μm, 80 μm, 90 μm, 100 μm, or 150 μm. The microparticle(s) may be at concentrations greater than 10 mg/mL, 20 mg/mL, 30 mg/mL, 40 mg/mL, 80 mg/mL, 90 mg/mL, 100 mg/mL, 150 mg/mL or 300 mg/mL, in a range between and including any two of 10 mg/mL, 20 mg/mL, 30 mg/mL, 40 mg/mL, 80 mg/mL, 90 mg/mL, 100 mg/mL, 150 mg/mL, 300 mg/mL or 450 mg/mL, or any one of 10 mg/mL, 20 mg/mL, 30 mg/mL, 40 mg/mL, 80 mg/mL, 90 mg/mL, 100 mg/mL, 150 mg/mL, 300 mg/mL, or 450 mg/mL.

### Embodiments List

This embodiment list of embodiments is not exhaustive of the embodiments herein.
1. A composition comprising a first fraction of hydrogel microparticles, where the hydrogel microparticles comprise an encapsulated collagen stimulatory agent.
2. The composition of embodiment 1, comprising at least one additional fraction of hydrogel microparticles, wherein at least two of the fractions of hydrogel microparticles have a different stability in aqueous buffer under physiological conditions.
3. The composition of embodiment 1 or 2, wherein all of the fractions of hydrogel microparticles have a different stability in aqueous buffer under physiological conditions.
4. The composition of embodiment 2 or 3, wherein the first fraction of hydrogel and the one or more of the at least one additional fraction of hydrogel microparticles comprise acrylate modified hyaluronic acid with an extent of acrylate modification between 2 and 20 percent, and are crosslinked with a thiol containing crosslinker.
5. The composition of embodiment 2 or 3, wherein the first fraction of hydrogel and the one or more of the at least one additional fraction of hydrogel microparticles comprise methacrylate modified hyaluronic acid with an extent of methacrylate modification between 2 and 20 percent, and are crosslinked with a thiol containing crosslinker.
6. The composition of any one of embodiments 2-5, wherein the stabilities differ by 7, 14, or 21 days or more.
7. The composition of embodiment 6, wherein one fraction selected from the first fraction or one of the at least one additional fraction has a stability of 30 days or less and another fraction selected from the first fraction or one of the at least one additional fraction has stability of 30 days or more, and the stability of the one fraction and the stability of the another fraction differ by 7, 14, 21 days or more.
8. The composition of any of embodiments 1-7, wherein the hydrogel microparticles are stable in aqueous buffer under physiological conditions for greater than 30 days.
9. The composition of any of embodiments 1-8, wherein the hydrogel microparticles are stable in aqueous buffer under physiological conditions for less than 90 days.
10. The composition of embodiment 1, wherein the first fraction of hydrogel microparticles comprises acrylate modified hyaluronic acid with an extent of acrylate modification between 2 and 20 percent, and is crosslinked with a thiol containing crosslinker.
11. The composition of embodiment 1, wherein the first fraction of hydrogel microparticles comprises methacrylate modified hyaluronic acid with an extent of methacrylate modification between 2 and 20 percent, and is crosslinked with a thiol containing crosslinker.
12. The composition of any one of embodiments 1-11, wherein the collagen stimulatory agent is in the form of a microparticle.
13. The composition of embodiment 12, wherein the microparticle of collagen stimulatory agent comprises poly(L-lactic acid).
14. A method of providing producing and maintaining an aesthetic feature comprising injecting the composition of any one of embodiments 1-13 into the skin of a subject.
15. The method of embodiment 14, wherein the subject is a mammal.
16. The method of embodiment 15, wherein the mammal is a human.
17. A method of producing or maintaining a volume in or under the skin comprising administering the composition of any one of embodiments 1-13 to the skin of a subject.
18. The method of embodiment 17, wherein the subject is a mammal.
19. The method of embodiment 18, wherein the mammal is a human.
20. The method of embodiment 17, wherein the administering is injecting into the skin.
21. The method of embodiment 17, wherein the injection is to a site within the subject where tissue loss is manifested.
22. A use of the composition of any one of embodiments 1-13 for producing and maintaining an aesthetic feature of the skin of a subject.
23. A use of the composition of any one of embodiments 1-13 for producing or maintaining a volume in or under the skin of a subject.

### EXAMPLES

### Example 1

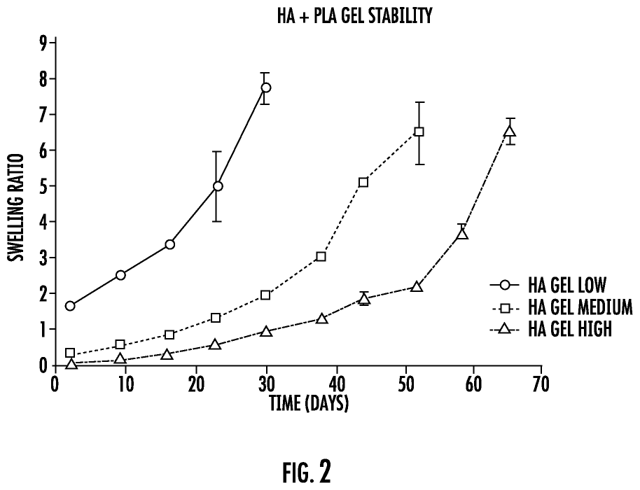

Sodium hyaluronate (700 kDa, Lifecore Biomedical) was chemically modified with an acrylate functional group by adding acrylic anhydride to an aqueous solution of sodium hyaluronate and maintaining the pH at 8.5 with NaOH for 18 hrs. The chemically modified HA was then purified and dried. The percent of disaccharides chemically modified with acrylate was determined with 1H NMR (Bruker 600 MHz). 2 wt % HA hydrogels were formed by mixing acrylated HA (4, 9 or 20 percent chemical modification corresponding to “Low”, “Medium” and “High”) with dithiothreitol at a molar ratio of 1:1 acrylate:thiol in PBS, adding the precursor solution to cylindrical molds and incubating at room temperature for three days. 50-100 μL hydrogels crosslinked in PBS were incubated in 1 mL PBS at 37° C. and hydrogel masses were measured overtime and buffer collected and replaced with fresh PBS. Swelling ratios were calculated by subtracting the original hydrogel mass from the hydrogel mass and dividing by the original hydrogel mass. Hydrogel masses were measured until the gel could not be separated from the buffer using a spatula or centrifugation at 2,000×g for 10 min, indicating complete dissolution (FIG. 2).

### Example 2

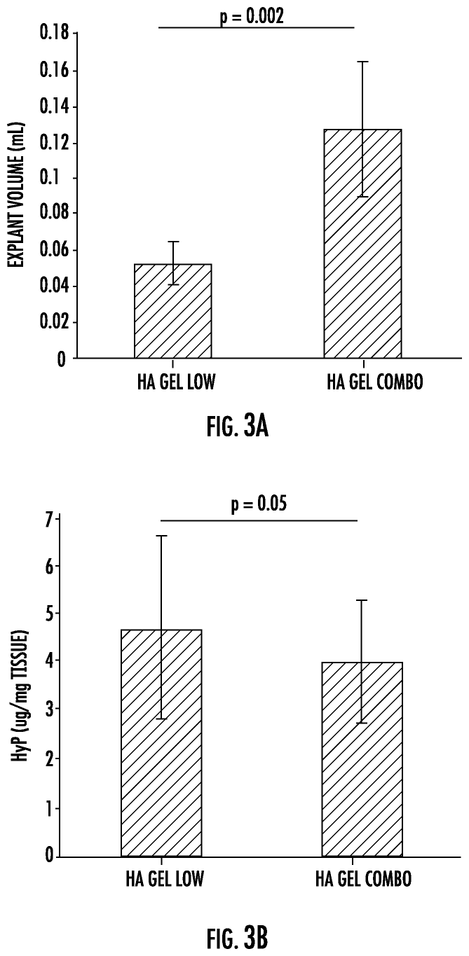

PLLA microparticles with diameters between 10 and 100 microns were be encapsulated at 75 mg/mL in the Low, Medium and High HA hydrogels described in Example 1. The hydrogels were then micronized by passing through screens with 595 and 330 micron mesh sizes, 5 times each. Hydrogel/PLLA microparticles from each of the Low Medium and High groups were mixed at equal masses to form a “Combo” group. 0.1 mL of the Low and Combo hydrogel/PLLA groups were injected subcutaneously in rats to produce an aesthetic feature. After 84 days, the rats were euthanized and aesthetic features/newly formed tissues explanted, dimensions measured and analyzed for hydroxyproline content using a hydroxyproline assay (Sigma) (FIG. 3).

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

1. A composition comprising a first fraction of hydrogel microparticles with encapsulated collagen stimulatory agent, wherein the hydrogel microparticles comprise ester crosslinks and are stable in aqueous buffer under physiological conditions for greater than 30 days.

2. The composition of claim 1, comprising at least one additional fraction of hydrogel microparticles, wherein at least two of the fractions of hydrogel microparticles have stabilities that differ by 7, 14, or 21 days or more in aqueous buffer under physiological conditions.

3. (canceled)

4. The composition of claim 1, wherein the hydrogel microparticles are made by crosslinking acrylate modified hyaluronic acid with an extent of acrylate modification between 0.1 and 20 percent.

5. (canceled)

6. (canceled)

7. (canceled)

8. (canceled)

9. The composition of claim 1, wherein the hydrogel microparticles are stable in aqueous buffer under physiological conditions for greater than 30 days and less than 180 days.

10. (canceled)

11. (canceled)

12. The composition of claim 1, wherein the collagen stimulatory agent comprises microparticles.

13. (canceled)

14. A method of providing, producing, and maintaining an aesthetic feature comprising injecting the composition of claim 1 into the skin of a subject.

15. (canceled)

16. (canceled)

17. A method of producing or maintaining a volume in or under the skin comprising administering the composition of claim 1 to the skin of a subject.

18. (canceled)

19. (canceled)

20. (canceled)

21. (canceled)

22. A use of the composition of claim 1 for producing and maintaining an aesthetic feature of the skin of a subject.

23. A use of the composition of claim 1 for producing or maintaining a volume in or under the skin of a subject.
